(19) United States
(12) Patent Application Publication
(10) Pub. No.: US 2024/0180158 A1
(43) Pub. Date: Jun. 6, 2024

(54) COMPOUNDS HAVING SELECTIVE INACTIVATION ACTIVITY

(71) Applicants: University of North Carolina at Wilmington, Wilmington, NC (US); THE UNIVERSITY OF TOLEDO, Toledo, OH (US)

(72) Inventors: Christopher John HALKIDES, Wilmington, NC (US); Ronald E. Viola, Toledo, OH (US)

(73) Assignees: University of North Carolina at Wilmington, Wilmington, NC (US); THE UNIVERSITY OF TOLEDO, Toledo, OH (US)

(21) Appl. No.: 18/417,805

(22) Filed: Jan. 19, 2024

Related U.S. Application Data
(63) Continuation-in-part of application No. PCT/US2022/037601, filed on Jul. 19, 2022.
(60) Provisional application No. 63/223,339, filed on Jul. 19, 2021.

(51) Int. Cl. A01N 43/42; A01N 31/04; A01N 37/06; A01N 37/22; A01N 43/16; A01P 1/00; A01P 3/00; C12N 9/99

(52) U.S. Cl. CPC A01N 43/42; A01N 31/04; A01N 37/06; A01N 37/22; A01N 43/16; A01P 1/00; A01P 3/00; C12N 9/99; C12Y 102/01031

(57) ABSTRACT

In one aspect, compounds and associated pharmaceutical compositions are described herein for the treatment of various fungal infections and/or other diseases. In some embodiments, a method for the inactivation of a microbial enzyme is provided comprising providing a compound having a selective inactivation activity, providing an enzyme target, and inactivating the enzyme target. In some example embodiments, the compound comprises a sulfonyl group.

### CROSS-REFERENCE TO RELATED APPLICATIONS

This application is a continuation-in-part of International Application No. PCT/US2022/037601, filed Jul. 19, 2022, which claims priority to U.S. Provisional Patent Application No. 63/223,339, filed Jul. 19, 2021, the contents of each being incorporated herein by reference in their entirety.

### FIELD

The technology described herein generally relates to antimicrobial compounds and modes of action associated with the compounds, more particularly to compounds that are selective inactivators against microbial enzyme targets and methods of selectively inactivating an enzyme target.

### BACKGROUND

Fungal infections are among the leading causes of human mortality, and this is particularly true in immunocompromised patients. For example, more than 20 different Candida species, including C. albicans, C. tropicalis, C. glabrata, C. krusei, and C. auris, can cause human diseases. Unfortunately, many of these Candida species already have natural resistance to some front-line antifungal drugs, and several are classified as multidrug-resistant emerging pathogenic threats. Antibiotic resistance has become the most significant health risk for infectious bacterial diseases, and this issue is even more acute for pathogenic fungal infections. There are significantly fewer effective antifungal drugs available for treatment, even before the emergence of resistance against these drugs. The paucity of antifungal drugs is due primarily to the significant overlap of pathways between humans and fungi, resulting in much greater challenges in identifying unique fungal drug targets. Despite these metabolic similarities among eukaryotic organisms there are some uniquely microbial metabolic pathways. The aspartate biosynthetic pathway is one such pathway, absent in mammals, but producing essential amino acids and other metabolites that are critical for microbial survival. Several of the genes in this pathway are found in the minimal set of essential genes required for microbial survival. This pathway has also recently been validated as an important new target for anti-tuberculosis drug development. Among the amino acid biosynthetic pathways only a single gene, the asd gene that codes for aspartate semialdehyde dehydrogenase (ASADH), has been shown to be essential in animal models for typhoid fever and enteritis. This same gene, called HOM2 in fungi, was identified by transcriptional repression to be among the 634 essential genes in C. albicans. Accordingly, there is a need to provide compounds which can be developed into effective new antimicrobial agents, for instance antifungal and/or antibacterial agents that will block this essential pathway by selectively inhibiting this validated drug target.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

At a high level, embodiments of the technology described herein are directed towards compounds having selective inactivation and/or inhibition activity, in particular when acting upon a microbial target, such as a microbial enzyme. In some embodiments, compounds described herein can be implemented to inactivate and/or selectively inactivate one or more target(s) or target compounds or target enzymes.

In one aspect, compounds and associated compositions are described herein for the treatment of various bacterial infections, fungal infections, and/or other diseases. In some embodiments, for example, compounds of Formula (I) are provided:

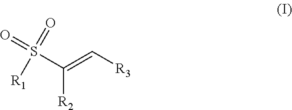

Wherein R₁, R₂, and R₃ are independently selected. According to some embodiments, such compounds have selective inactivation properties against or in the presence of one or more enzymes.

In some instances, R₁ can be, but not limited to, a methyl, phenyl, or trifluoromethyl functional group. R₂ can be a hydrogen group or other simple functional group, and R₃ can be selected from a variety of functional groups, for example an alkyl, aryl, substituted aryl, heteroaryl, or amino acid based group.

In another aspect, methods of treating bacterial infections are described herein. In some embodiments, a method comprises administering to a patient having a bacterial or fungal infection a therapeutically effective amount of one or more compounds of Formula (I).

In some embodiments, a method of implementing a compound having selective inactivation energy is provided. In an embodiment, a method comprises providing a compound having a selective inactivation activity, providing an enzyme target, and inactivating the enzyme target with the compound having the selective inactivation activity. In some embodiments, the compound comprises a sulfonyl group. In some embodiments, the compound comprises a vinyl sulfone or a vinyl sulfonamide. In some embodiments, the compound comprises a carboxyl group and/or a nitro group. In some embodiments, the compound exhibits antifungal and/or antibacterial properties. In some embodiments, the enzyme target is a microbial enzyme target, for example ASADH. In some embodiments, when a compound comprises a vinyl sulfone or a vinyl sulfonamide, the vinyl sulfone or vinyl sulfonamide is configured as an isotere of a mixed phosphoric carboxylic anhydride. In some embodiments, the compound can be configured to match a binding pocket of a microbial enzyme target, such as ASADH. In some other embodiments, the compound is a sulfonyl keytone or a sulfonyl acrylamide. In some embodiments, the enzyme target is irreversibly inhibited, or for example irreversibly inhibited by a covalent bonding mechanism.

These and other embodiments are further described in the following detailed description. It will be appreciated that additional objects, advantages, and novel features of the invention will be set forth in part in the following description.

### BRIEF DESCRIPTION OF THE DRAWINGS

Aspects of the technology presented herein are described in detail below with reference to the accompanying figures, wherein:

FIG. 1 shows molecular modeling and docking of an example of a substituted aryl vinyl sulfone into the active site of a fungal ASADH, in accordance with some embodiments of the present technology;

FIG. 2 shows molecular modeling and docking of an example of an aryl vinyl sulfone into the active site of a fungal ASADH, in accordance with some embodiments of the present technology;

FIG. 3 illustrates the kinetics of the inactivation of a fungal ASADH by an example vinyl sulfone, in accordance with some embodiments of the present technology;

FIG. 4 is a table showing docking of example aryl vinyl sulfones to a fungal ASADH and calculations of their expected affinities, in accordance with some embodiments of the present technology;

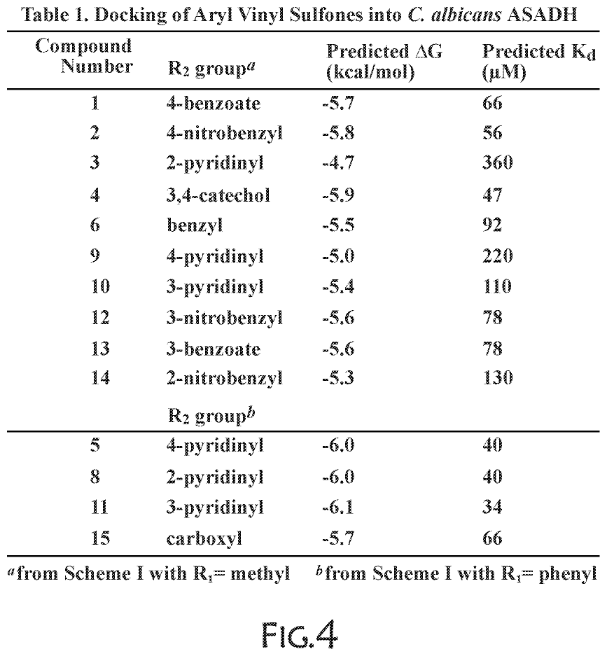

FIG. 5 is a table showing the affinities and the rates of inactivation of example vinyl sulfones as inactivators of a fungal ASADH, in accordance with some embodiments of the present technology;

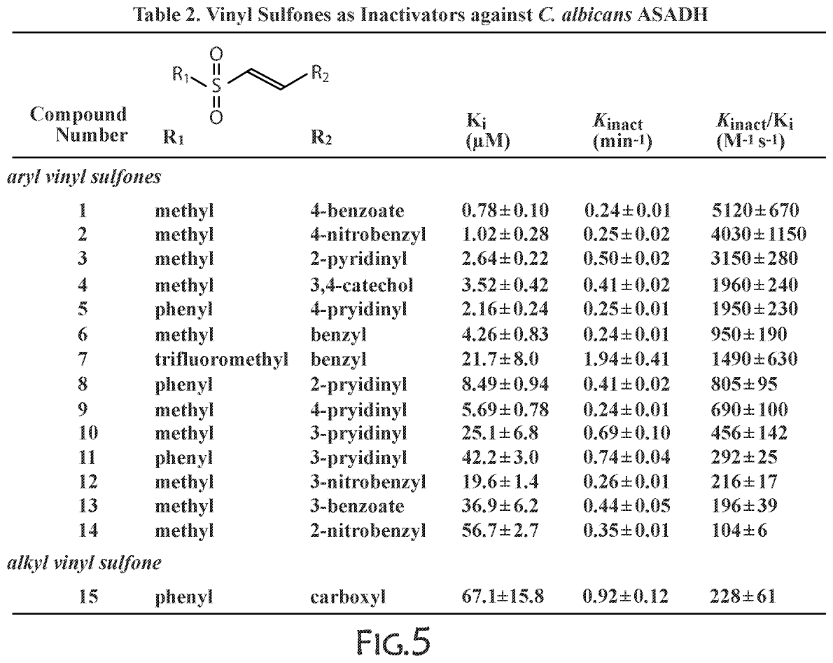

FIG. 6 is a table showing a comparison of the differences in affinities and rates of inactivation of example vinyl sulfones as inactivators of either a bacterial or a fungal ASADH, in accordance with some embodiments of the present technology; and

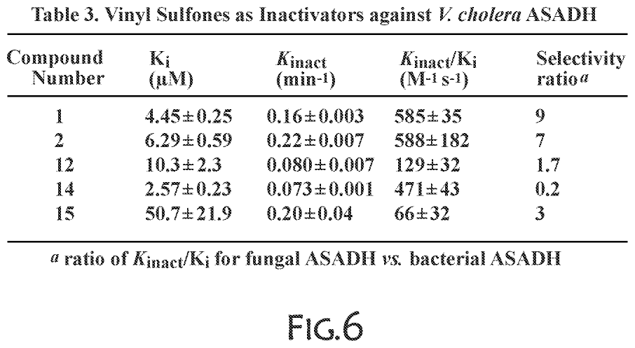

FIG. 7 illustrates an example mechanism of inactivation and/or inhibition, in accordance with some embodiments of the present technology.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

Reasonably potent reversible inhibitors have been developed against bacterial forms of ASADH by using a structure-guided approach to elaborate initial hits from fragment library. More recently, reversible inhibitors have been identified which show some selectivity towards fungal ASADHs. While promising, these compounds still need further optimization to achieve sufficient potency and selectivity to begin pre-clinical evaluation.

This alternative approach to traditional drug development is aimed at designing compounds that will selectively inactivate their target. The vast majority of drugs that have been introduced into clinical use were not designed to function as irreversible inactivators. Yet about 20-30% of the drugs that target enzymes, including commonly used drugs such as aspirin and penicillin, have subsequently been shown to function by covalently modifying their respective targets. Despite these successes there is still a prevailing prejudice that enzyme inactivators are considerably less likely to possess desirable drug-like properties. There are two legitimate concerns that could limit the potential applications of this class of drugs: (1) highly reactive functional groups are typically not selective, and (2) less reactive functional groups may not have sufficient potency to be effective. Finding a balance between these two unproductive limiting cases is challenging. This challenge can be overcome if these two desirable properties, reactivity and selectivity, can be separately adjusted for a target of interest.

According to aspects of the present technology, vinyl sulfones are a reactive functional group that are susceptible to nucleophilic attack to form covalent adducts (e.g. Scheme I). Vinyl sulfones are also a class of compounds that meet the definition of “quiescent affinity labels.” This class of compounds are known to be unreactive in the presence of most biologically-relevant nucleophiles, but react readily when bound and properly oriented in a site with a complementary surface containing appropriately positioned activating groups. Furthermore, the reactivity of vinyl sulfones can potentially be altered by changing the nature of the adjacent groups. Their target affinity and selectivity can also be improved through the introduction of complementary binding groups that mimic those of the natural enzyme substrates.

According to some aspects, an inhibitor development approach is initiated through the design and synthesis of selective enzyme inactivators with the goal of producing potent compounds that will target fungal ASADHs. The reactive vinyl sulfone functional group can potentially act as a mimic of the phosphate ester of the aspartyl phosphate substrate of ASADH. According to the present technology, vinyl sulphones and their design and synthesis demonstrate excellent potency as covalent, irreversible inactivators of fungal and/or bacterial agents, for example Candida albicans ASADH (CalASADH).

Generally, embodiments of the technology described herein are compounds based on the structure of Formula (I). In some instances, R₁ can be a methyl, phenyl, or trifluoromethyl functional group. In some instances, R₂ can be a hydrogen group. In some instances, R₃ can be an alkyl, aryl, substituted aryl, heteroaryl, or amino acid based group or derivative thereof, such as CH₂CH(NH₂)CO₂H. In some further instances, R₁ can be a benzyl (e.g. PhCH₂) or a 2-pyridyl. According to embodiments of the present technology, one validated target for antimicrobials is aspartate semialdehyde dehydrogenase (ASADH) which is an enzyme needed to produce various components of some bacterial cell walls.

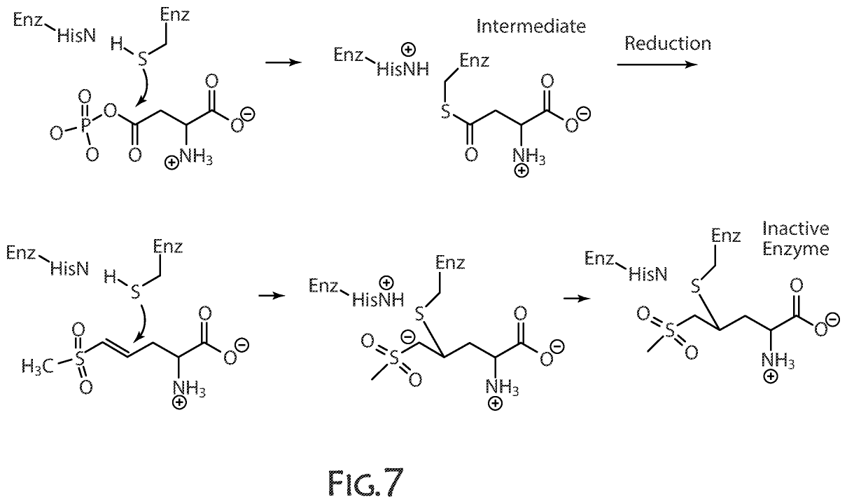

Accordingly, compounds described herein can inhibit the enzyme aspartate of β-semialdehyde dehydrogenase (ASADH) in an irreversible manner with good affinity by forming a covalent bond, for instance to the cysteine residue (cys residue) that is the nucleophile in a reaction that ASADH catalyzes. It will be appreciated that ASADH is an enzyme in the biosynthetic pathway to lysine, threonine, isoleucine, and methionine, among other molecules. It is a validated target for antimicrobial compounds, for instance with respect to bacteria and fungi. Because ASADH is not found in mammals, inhibitors of this enzyme are less likely to be toxic to mammals. Referring briefly to FIG. 7, an example mechanism of inhibition and/or inactivation is illustrated. In a first step of the reaction, the cysteine residue of the ASADH may attack the carbonyl carbon of a mixed anhydride to form a thioester, and histidine is the general base. Subsequently, NADPH reduces the thioester. In an inactivation step, the cys residue of ASADH attacks the vinyl group to produce a carbanion. In another inactivation step the histidine residue protonates carbon, producing an inactive form of ASADH.

### I. Compounds and Pharmaceutical Compositions Having Antimicrobial Activity

Various compounds are described herein, as well as the preparation and use thereof. As discussed above, and further illustrated in the examples below, the compounds can exhibit antimicrobial properties in some embodiments, for instance antifungal and antibacterial properties. Some embodiments described herein are further illustrated in the following non-limiting examples.

Accordingly, the design of highly selective enzyme inactivators against a validated drug target has the potential to produce a completely new class of antimicrobial and/or antifungal agents.

A set of aryl vinyl sulfones were synthesized with different aromatic or substituted aromatic groups to determine whether these compounds could be induced to function as effective inactivators of fungal ASADH. Most of these aryl vinyl sulfones were synthesized through the coupling of aryl aldehydes with methylsulfonyl phosphonates (Scheme II), where R₁ is either a methyl or a phenyl group. Some polar (NO₂) or charged (COO⁻) functional groups were positioned around the aryl ring, guided by the modeling studies described above which suggested the possibility of making specific interactions in the active site of ASADH. A set of pyridinyl vinyl sulfones were also synthesized by the same approach to examine the impact of orienting a heteroaromatic ring that would be more electron-withdrawing into this series of vinyl sulfones. In addition, a catechol-containing vinyl sulfone was synthesized (Scheme III) to assess the role of hydroxyl groups as potential hydrogen-bonding partners to improve binding affinity.

Each of the synthesized aryl vinyl sulfones that incorporate these additional functional groups were found to inhibit the ASADH purified from the pathogenic fungal organism Candida albicans (CalASADH). Their initial inhibition potencies (Kᵢ values) were determined by varying the concentration of each vinyl sulfone in the presence of fixed, non-saturating substrate levels. In contrast to the low affinity of simple vinyl sulfones which possess Kᵢ values in the low millimolar range, these synthesized aryl vinyl sulfones each have substantially improved Kᵢ values. The most potent inhibitors achieved affinities towards CalASADH in the very low to sub-micromolar range (e.g. Table 2).

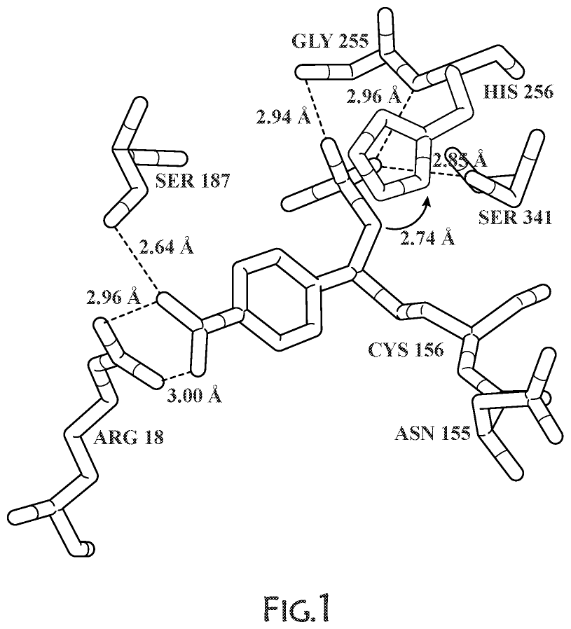

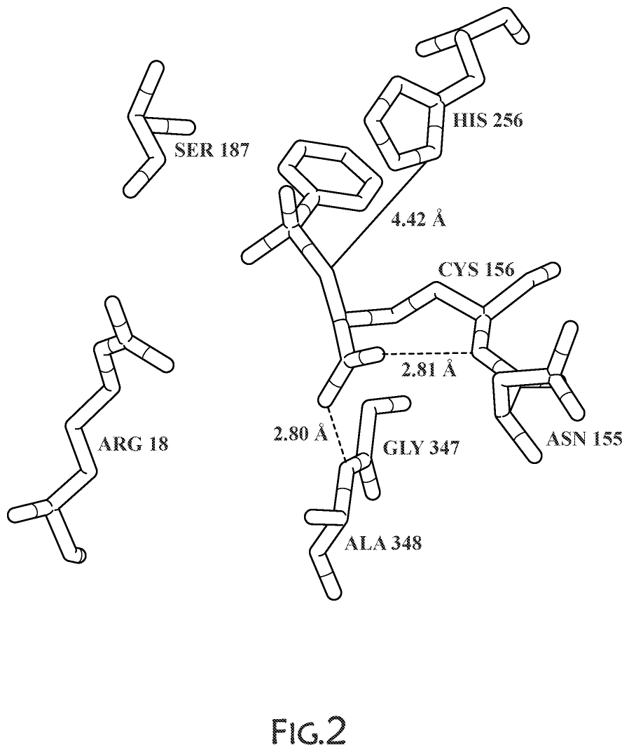

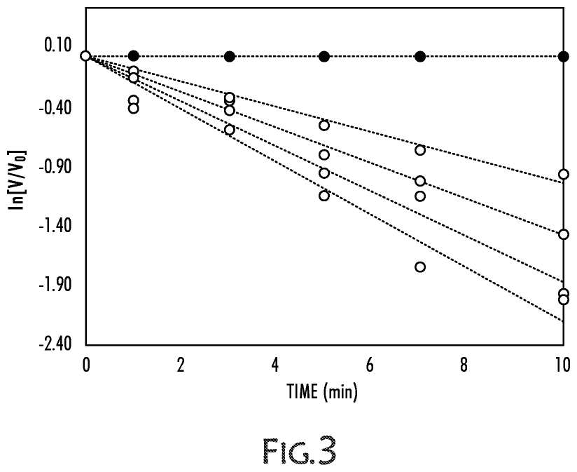

Aryl vinyl sulfones can be reasonably potent inhibitors of CalASADH, each inhibitor was then tested to determine if it would function as an enzyme inactivator. Pre-incubation studies of each synthesized compound with this enzyme revealed that these compounds can also function as time-dependent inactivators. In most cases the residual enzyme activity, measured following a subsequent 20-fold dilution into an assay mixture, decreased to less than 5% of the control rate after incubation with low micromolar levels of these inactivators for 10 min or less. To determine the rate of inactivation the concentration of each vinyl sulfone in this pre-incubation mixture was then varied, centered around their initial measured Kᵢ values. The rate of activity loss observed in each incubation reaction (k_{obs}) was plotted against the inhibitor concentration to determine the inactivation rate (k_{inact}) for that compound and the concentration that produces half-maximal inactivation (Kᵢ). An example of this inactivation is shown (FIG. 3) with the concentration of 4-(methylvinylsulfonyl)nitrobenzene varied from 0.75 to 6 μM for an effective compound that had been suggested by modeling to dock into the active site of CalASADH (FIG. 1). Turning briefly to the figures, FIG. 1 shows molecular modeling and docking of 4-(methylsulfonylvinyl)nitrobenzene into the active site of Candida albicans ASADH. The proposed product of the reaction of the active site nucleophile (Cys-156) with this inactivator predicts key interactions of the p-nitro group with Arg-18 and Ser-187, and the protonation of the covalent intermediate by His-256. FIG. 2 shows molecular modeling and docking of 3-(phenylsulfonyl)acrylic acid into the active site of Candida albicans ASADH. Reaction of this inactivator with the active site nucleophile (Cys-156) predicts keys interactions between its carboxyl group and several backbone amide nitrogens that would shift this adduct further from the active site base (His-256). FIG. 3 illustrates inactivation of Candida albicans ASADH by 4-(methylvinylsulfonyl)nitrobenzene. Plot of the natural log of the ratio of enzyme activity at each time point over the initial enzyme activity, showing the loss of activity as a functional of incubation time for different concentrations of inactivator: 0 μM, 0.75 μM, 1.5 μM, 3 μM, 6 μM. The resulting observed rates at each concentration (k_{obs}) were plotted against the inactivator concentration and a non-linear fit to these replotted data yielded the rate of inactivation (k_{inact}) and the inhibition constant (Kᵢ).

To enhance the reactivity of these vinyl sulfones towards CalASADH a series of pyridinyl vinyl sulfones were synthesized with the methyl group at position R₁ replaced by a phenyl group. Several of these phenyl derivatives did show improved affinity towards ASADH, but the rates of inactivation of this enzyme were unaffected by this substitution (Table 2). However, when a trifluoromethyl group was introduced at this position in the benzyl vinyl sulfone that rate of inactivation is enhanced by 8-fold relative to the corresponding inactivator with a methyl group (Table 2).

Alkyl vinyl sulfones can also act as an ASADH inactivator. Docking studies reveal a number of potential interactions within the substrate binding pocket that offer the possibility of improving the binding and orientation of even simple alkyl vinyl sulfones with appropriately placed functional groups to serve as ASADH inactivators. To test this possibility a carboxyl-containing alkyl vinyl sulfone was synthesized through a coupling and debromination reaction (Scheme IV). The resulting acrylic acid derivative is a strong inhibitor, with a Kᵢ value that is enhanced by greater than 10-fold when compared to those of the simple methyl and ethyl vinyl sulfones. This new compound (where R₁=phenyl and R₂=carboxyl) has a low micromolar Kᵢ value and also show a faster rate of inactivation of C. albicans ASADH when compared to the rates for the aryl vinyl sulfones (Table 2).

Samples of ASADH which were pre-incubated with a series of these vinyl sulfones failed to recover catalytic activity after being diluted into an assay mixture. This loss of activity supports the hypothesis that these vinyl sulfones are covalently modifying this enzyme. To determine whether this inactivation can be reversed by the introduction of an exogenous nucleophile a large excess of either cysteine (5 mM) or glutathione (10 mM) was added to the reaction mixture after incubation, and the activity of this inactivated enzyme was then monitored. Samples of CalASADH that had been incubated either with 3 μM 4-(methylvinylsulfonyl)nitrobenzene or with 100 μM 3-(phenylsulfonyl)acrylic acid for 10 min were measured to have from 5 to 10% residual activity. At this point, after either cysteine or glutathione was added to each reaction mixture, the residual enzyme activity was monitored as described above. No increase in enzyme activity was observed over a time period from 30 min to 4 h in the presence of either thiol reagent, supporting the irreversible inactivation model for both an aryl and an alkyl vinyl sulfone (Scheme I).

Enhanced reactivity upon binding to an enzyme target is a desirable property if these vinyl sulfones are to serve as effective antifungal agents. However, increased selectivity in target binding is equally important. As a stringent test of specificity, several aryl vinyl sulfones that have been shown to function as potent inactivators of CalASADH were next examined against a bacterial ASADH ortholog. The ASADH from the Gram-positive bacterium Vibrio cholera has >90% overall sequence homology to the C. albicans enzyme, with the exception of a helical domain insert (Arachea et al., 2010), and all of the essential active site functional amino acids are fully conserved between these enzyme forms. Five different vinyl sulfones that span the fungal ASADH potency spectrum were selected and examined as potential inactivators of the bacterial ASADH. Each of these compounds were also found to inactivate this bacterial ortholog with low micromolar affinities, and with rates of inactivation ranging from 0.07 to 0.2 per min. However, importantly, several of these compounds already show some level of discrimination between the fungal and bacterial ASADHs (Table 3).

According to some embodiments, vinyl sulfone compounds contain both polar and, in some cases, charged functional groups that would make cellular uptake more challenging. However, to test the possibility that these compounds could inhibit the growth of C. albicans, two inactivators were selected for examination. Growing C. albicans cells were exposed for 1 h to several concentrations of a very good ASADH inactivator (4-(methylsulfonylvinyl)benzoic acid) and a moderate inactivator (4-(phenylsulfonylvinyl)pyridine). The cells were plated and their survival rates were measured after 48 h relative to a control growth in the absence of inactivator. In each case some decrease in cell survival rate was observed, especially at the highest concentration tested. Also, the more potent ASADH inactivator had a greater effect on cell survival, with less than 75% survival at 25 μM compound exposure.

The affinity that a reversible (or an irreversible) inhibitor exhibits towards its target enzyme (Kᵢ value) is an important parameter that is typically used as the primary criterion for measuring inhibitor potency. Low to sub-nanomolar Kᵢ values is the typical goal to be achieved for producing potent drug candidates. For inactivators that form a covalent bond to inactivate their target the rate of this inactivation (k_{inact}) also serves as a critical measure of compound potency. The ratio of these values (k_{inact}/Kᵢ) is the rate constant that describes the efficiency of covalent bond formation. Optimization of this ratio can be used to guide improvements in both efficacy and selectivity of covalent drug candidates.

According to some embodiments, enzyme inactivators have advantages over enzyme inhibitors. compounds that can selectivity bind to an essential enzyme target and block its activity is an important first step for the development of new antifungal agents to combat the growing threats posed by drug-resistant fungal species. Unlike the reversible binding of enzyme inhibitors, where the inhibition can be overcome by increasing substrate concentrations, the activity of an enzyme blocked by irreversible covalent inactivation can only be overcome through the production of new enzyme. This mode of action offers a significant advantage as a potential treatment against fungal infections, and there is a growing recognition of the value of developing these types of covalent drug candidates.

The most severe criticism of covalent inactivators as potential drugs is the concern that these reactive compounds will not be sufficiently selective against only the intended target. Identifying compounds whose reactivity is enhanced upon binding to its target, thereby leading to irreversible inactivation of this enzyme, would be a significant added bonus towards achieving target selectivity. Vinyl sulfones are potentially reactive functional groups that are susceptible to nucleophilic attack (Scheme I), but this class of compounds are not particularly reactive in the presence of most biological nucleophiles. For example, peptidyl vinyl sulfones designed to inactivate cysteine proteases showed only minimal reactivity after extensive incubation with excess glutathione. This low inherent reactivity has been confirmed with several simple vinyl sulfones that were tested against CalASADH and were found to either not inactivate this enzyme or led to inactivation only when examined at very high levels. These results confirm that vinyl sulfones do not have high inherent reactivity. This class of compounds is unlikely to serve as general inactivators of cysteine nucleophilic enzymes, or other potential targets, unless some level of binding specificity is incorporated into their structures.

Vinyl sulfones as described herein can provide target enzyme inactivation. Favorable binding interactions are present between specifically designed vinyl sulfones and some active site functional groups in fungal ASADHs (FIGS. 1 and 2), and as such synthesized aryl vinyl sulfones can function as potent inactivators of our target enzyme. The majority of these compounds bind to CalASADH with very low micromolar affinities and inactivate this enzyme with significant rates, ranging from 0.2 to 0.7 per min (Table 2).

Without being bound by theory, the inherent reactivity of vinyl sulfones towards nucleophilic attack can be enhanced by introducing more electron-withdrawing groups at the R₁ or R₂ positions. While there were no changes in the rates of inactivation when the methyl group at position R₁ was replaced by a phenyl group, the substitution of a more electron-withdrawing trifluoromethyl group did result in a significant rate enhancement.

The rates of inactivation for these compounds are already significantly faster than those reported for the best vinyl sulfone inactivators of protein tyrosine phosphatases. When the efficiency of covalent bond formation (k_{inact}/Kᵢ) is used as the evaluation criterion, the best CalASADH inactivators have values for this ratio that approach 10⁴ (Table 2). These values for our ASADH inactivators already compare quite favorably to the values observed for several other classes of covalent drugs and drug candidates.

Ideally, any antifungal agent should have high specificity, interacting only with its intended target. Despite their failure to function as potent inactivators of ASADH, some simple vinyl sulfones were shown to inactivate protein tyrosine phosphatases through covalent modification of its active site cysteine. This discrimination suggests some level of selectivity between enzymes that utilize active site cysteines as nucleophiles. A greater challenge would be to demonstrate species-selectivity between the same enzyme that was isolated from different organisms. Each of the vinyl sulfones that were examined as inactivators of a fungal ASADH were also found to inactivate the bacterial ASADH ortholog, but several of them do so with lower efficiencies. In particular, the best fungal ASADH inactivators with a polar functional group at the 4-position of the benzyl ring are from 7 to 9-fold less efficient when examined as bacterial ASADH inactivators (See e.g. Table 3). By contrast, the inactivators with this polar group in the 2- or 3-position show no significant differences in reactivity between the fungal and bacterial forms of ASADH. The respective active sites of these two ASADH orthologs are virtually identical. However, a conserved arginine (Arg-18) found in fungal ASADH that is proposed to interact with the 4-nitro or the 4-carboxy groups in these inactivators (FIG. 1) is replaced with either a valine or a threonine in the bacterial ASADH family. This mutation would explain the lower efficacy with the V. cholera ASADH, and suggests that the 4-substituted benzyl group is an important structural element which must be retained during the optimization of this series of enzyme inactivators.

The docking of aryl vinyl sulfones into the active site of CalASADH proposes the proximity of a base (His-256) in a suitable position to protonate the initially-formed covalent adduct with the active site nucleophile (Cys-156) and lead to irreversible enzyme inactivation (Scheme I). The failure of a vast excess of two different thiol-containing reagents to displace this modifying group and reactivate the enzyme provides evidence in support of this inactivation mechanism. The predicted docking of an alkyl vinyl sulfone was less definitive in supporting the potential involvement of this base in this irreversible inactivation mechanism, because of the greater distance that would be involved in proton transfer (FIG. 2). Nevertheless, exogenous thiols were still unsuccessful in reactivating CalASADH that had been treated with an alkyl vinyl sulfone, thereby supporting a similar mechanism of irreversible inactivation. For instance, exposing growth C. albicans cells to several different vinyl sulfones led to a decrease in cell survival. This shows that even these quite polar compounds can gain some access to fungal cells and inhibit their growth.

### EXAMPLES

### Compound Classes for Enzyme Inactivation

Compounds and associated compositions are described herein for the treatment of various bacterial infections, fungal infections, and/or other diseases, and can for example exhibit inactivation characteristics against one or more enzymes. In some embodiments, for example, compounds described herein can correspond to one of the classes or structures or structural formulas shown below:

| Compound Class | Structure |
|---|---|
| (1) Vinyl sulfone | 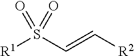 |
| (2) Vinyl sulfonamide | 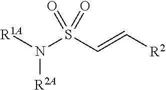 |
| (3) Acrylamide | 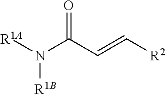 |
| (4) Sulfonyl ketone (keto sulfone) | 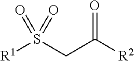 |
| (5) Sulfonyl acrylamide (carboxamido vinyl sulfone) | 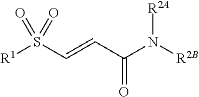 |

As indicated above, various classes of compounds having inactivation activity are provided. In some embodiments, examples of R¹ include, but are not limited to, methyl, phenyl, 2-pyridyl, benzyl, propyl, and cyclopropyl. In some embodiments, examples of R² include, but are not limited to, nitrophenyl, pyridyl, phenyl, nitrothiophenyl, carboxyphenyl, and alanyl. In some embodiments, examples of R^{1A} and R^{1B} include but are not limited to hydrogen, methyl, and the rest of the morpholine ring: —CH₂CH₂OCH₂CH₂. In some embodiments, examples of R^{2A} and R^{2B} include but are not limited to hydrogen, methyl, and the rest of the morpholine ring: —CH₂CH₂OCH₂CH₂.

In some aspects, as will be appreciated, and in light of embodiments described herein, classes (2)-(5) can in some instances be considered as subclasses of class (1). Accordingly, in some embodiments, R¹ and/or R² comprise substituted amines or amides. In some embodiments R¹ comprises any alkyl, vinyl, aryl, heteroaryl, or amino groups. In some embodiments, R² comprises any alkyl, vinyl, aryl, heteroaryl, or carboxamido groups. For example, R¹ can include, but is not limited to, methyl, phenyl, 2-pyridyl, benzyl, propyl, cyclopropyl, and dimethylamino. Further, for example R² can include, but is not limited to nitrophenyl, pyridyl, phenyl, nitrothiophenyl, carboxyphenyl, alanyl, and C(O)NRR, where R can be alkyl, aryl, heteroaryl or H.

The following examples are in light of the above-identified classes of inactivation compounds and further illustrate methods of making corresponding example compounds and properties thereof.

### Vinyl Sulfonamide.

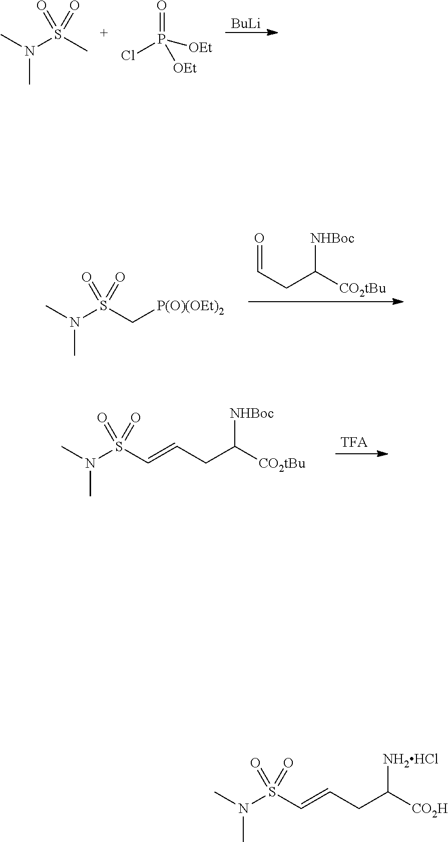

A solution of N,N-dimethyl methanesulfonamide (0.503 g, 4.1 mmol) was prepared in dry THF and cooled to −70° C. A solution of n-BuLi (2.8 mL, 4.5 mmol?) 1.6 M in hexanes was added dropwise with stirring over 15 minutes. The reaction was brought to −40° C. for two hours then returned to −70° C. A solution of diethoxychlorophosphate (7.00 g. 4.1 mmol) in THF (4.1 mL) was added dropwise. The reaction was allowed to come to room temperature and stirred for several hours before being stored at 5° C. overnight. To the reaction mixture was added a 15-mL aliquot of water, and the product was extracted with four 15-mL portions of DCM, drying the organic layer with sodium sulfate. The volatiles were removed, and the crude product was purified over silica using 70:30 to 100:0 ethyl acetate/hexanes. The yield was 0.32 grams (30%). H-1 NMR: 4.26 (4H, m), 3.56 (2H, d, J=17.1), 2.96 (6H, s), 1.40 (t, J=7.2). C-13 NMR: 63.44 (d, J=6.6) 45.54 (d, J=139.8), 37.65 (s), 16.34 (d, J=6.3). P-31 NMR: 13.3 ppm (s).

The compound tert-butyl 2-((tert-butoxycarbonyl)amino)-4-oxobutanoate is abbreviated as Boc-Asp(H)-OtBu below, and it was synthesized. A Homer Wadsworth Emmons reaction was performed as described with minor modifications. Lithium chloride (0.070 g, 1.66 mmol) was dissolved in acetonitrile (8 mL) with stirring. Solutions of diethyl N,N-dimethylsulfonamidomethylphosphonate (0.259 g, 0.998 mmol), DBU (0.138 g, 0.907 mmol), and Boc-Asp(H)-OtBu (0.248 g, 0.908 mmol) in acetonitrile were added in succession. The reaction was followed to completion by TLC and quenched with saturated ammonium chloride. After the acetonitrile was removed by rotary evaporation, a small portion of water was added, and the product was extracted four times with DCM. The combined DCM layers were dried with MgSO₄, and the DCM was removed by rotary evaporation. The product was eluted from silica using 35:65 ethyl acetate/hexanes. H-1 (CDCl₃): 6.67 (m, 1H), 6.21 (m, 1H), 5.20 (m, 1H), 4.37 (m, 1H), 2.79 (s, 6H), 2.69 (m, 2H), 1.50 (s, 9H), 1.47 (s, 9H). C-13 (CDCl₃): 170.05, 155.02, 141.52, 126.40, 83.12, 82.78, 80.23, 52.86, 37.52, 34.84, 28.32, 28.04.

The protected amino acid bearing a vinyl sulfonamide from the previous step was stirred in 4:1 TFA/DCM for sixteen hours. The volatiles were removed with rotary evaporation. Then two small portions of toluene were added and removed with rotary evaporation, and the sample placed on high vacuum. The product was dissolved in water (0.5 column volumes) and applied to a 20-fold excess of Dowex AG-50(H⁺). The column was rinsed with 1.5-column volumes of water, and the sample was eluted with 4.0 column volumes of 2 M HCl. The solvent was removed with rotary evaporation and high vacuum. H-1 NMR (d₆-DMSO): 13.94 (s, 1H), 8.63 (s, 3H), 6.65 (m, 2H), 4.19 (m, 1H), 2.80 (m, 2H), 2.66 (s, 3H). C-13 NMR (d₆-DMSO): 170.43, 140.76, 127.20, 51.23, 37.70, 32.19.

### Acrylamide.

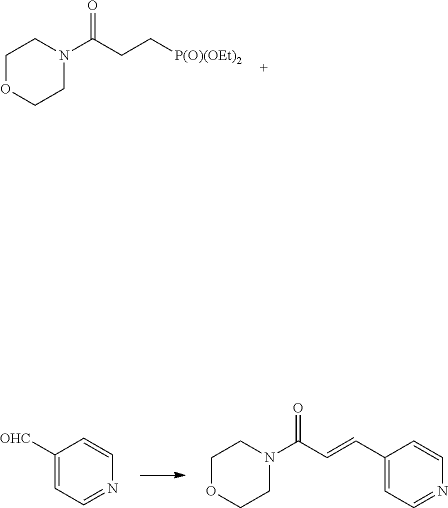

Diethyl α-morpholinecarboxamidomethylphosphonate. Triethylphosphite (1.88 g, 11.3 mmol) and 4-(2-chloroacetyl)morphine (2.00 g, 12.2) were stirred in a flask fitted with a reflux condenser and heated to 125° C. The reaction was monitored to completion (16 hours) by P-31 NMR. The crude product was applied to silica and eluted with 90:10 ethyl acetate/ethanol. The yield was 1.62 g (57%). H-1 NMR: 4.19 (m, 4H), 3.67 (m, 8H), 3.11 (d, J=N/A, 2H), 1.36 (t, J=N/A, 6H). C-13 NMR: 163.38, 66.73, 62.76, 47.34, 42.41, 33.26, 16.37.

4-morpholinecarboxyamidovinylpyridine. A protocol for the Homer Wadsworth Emmons reaction was followed with modifications. Lithium chloride (0.184 g, 3.42 mmol) was dissolved in acetonitrile (20 mL) with stirring. Solutions of Diethyl α-morpholinecarboxamidomethylphosphonate (0.545 g, 2.06 mmol), DBU (0.299 g, 1.87 mmol), and 4-formylpyridine (0.199 g, 2.06 mmol) in acetonitrile were added in succession. The reaction was followed to completion by TLC and quenched with saturated ammonium chloride. After the acetonitrile was removed by rotary evaporation, a small portion of water was added and the product extracted four times with DCM. The combined DCM layers were dried with MgSO₄, and the DCM was removed by rotary evaporation. The product was eluted from silica with 95:5 acetone/methanol, which was removed to provide 0.34 grams of the final product. H-1: 8.67, 7.62 (d, J=15.6, 1H), 7.42 (m, 2H), 7.05 (d, J=15.6, 1H), 3.75 (m, 8H). C-13: 164.44, 149.89, 143.03, 140.05, 121.90, 121.72, 66.81, 66.74, 47.32, 46.36.

Sulfonyl ketone.

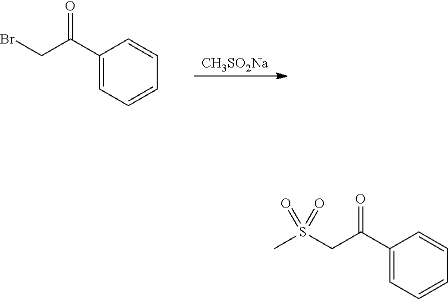

Methylsulfonylacetophenone. Sodium Methane sulfinate (0.223 g, 2.282 mmol) and a 10-ml aliquot of CH₃CN were added to a reaction flask and stirred. Phenacyl Bromide (0.407 g, 2.0447 mmol) and then tetrabutylammonium bromide (0.0660 g, 0.02047 mmol) were added to the reaction flask. The reaction was followed to completion by TLC (about 2 hrs). The solvent was removed using rotary evaporation, and the product was extracted using ethyl acetate and water. The combined ethyl acetate layers were dried with sodium sulfate, and the solvent was removed under reduced pressure. The product was eluted from silica using 50:50 EtOAc/Hexanes. The yield was 0.240 g (59%). H-1 NMR (CDCl₃): 8.04 (m, 2H), 7.76 (m, 1H), 7.54 (m, 2H), 4.63 (s, 2H), 3.18 (s, 3H). C-13 NMR (CDCl₃): 189.23, 135.61, 134.77, 129.27, 129.08, 61.29, 41.83.

### Sulfonyl Acrylamide.

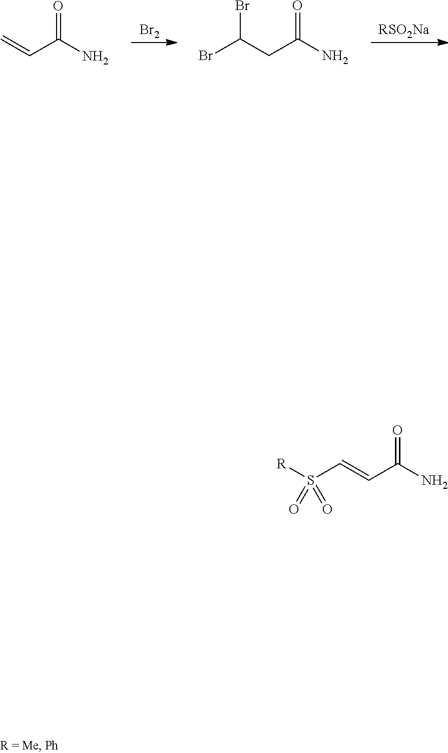

Acrylamide (1.02 g, 14.4 mmol) was dissolved in methanol (6 mL), and bromine (2.25 g, 14.1 mmol) was added dropwise with stirring. The dropping funnel was rinsed with a 1-mL portion of methanol. The reaction was refluxed for 2.5 hours, until most of the orange color had faded. Methanol was removed by rotary evaporation. The product was recrystallized from ethanol. 2,3-dibromopropionamide (0.403 g, 1.74 mmol) and sodium phenylsulfinate (0.430 g, 2.62 mmol) were dissolved in a 3.5 mL portion of DMF and heated with stirring to 80-90° C. for 24 hours. DMF was removed by adding heptane and performing rotary evaporation twice. The product was extracted into ethyl acetate three times from a small portion of water. The combined organic layers were dried with sodium sulfate, and the volatiles were removed with rotary evaporation. The crude product was eluted from silica with ethyl acetate, which was removed with rotary evaporation, leaving 0.113 g of product (31%). H-1 (d₆-DMSO): 8.04 (s, 1H), 7.99-7.82 (m, 2H), 7.87-7.56 (m, 4H), 7.46 (d, J=15.0 Hz, 1H), 6.99 (d, J=15.0 Hz, 1H), 3.33 (s, 3H), 2.55-2.45 (m, 3H). C-13 (d₆-DMSO): 163.53, 139.49, 139.35, 135.83, 134.83, 130.30, 128.17.

In some example embodiments, enzymes such as ASADH can be inhibited by one or more mechanisms via a sulfonyl ketone, for instance. Sulfonyl ketones may inhibit aspartate semilaldehyde dehydrogenase (ASADH) by at least two mechanisms.

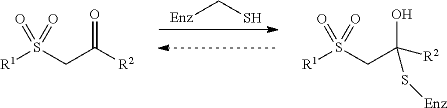

A first example mechanism shown above is that the essential cysteine residue adds into the carbonyl carbon of the ketone group, to create a hemithioketal. Enz refers to the enzyme ASADH, and SH refers to the thiol group of the essential cysteine. The ketone group in the inhibitor is expected to occupy the same position as the carbonyl group of the substrate, aspartyl phosphate. This class of compounds may be either reversible or irreversible, covalent inhibitors.

A second example mechanism shown below is that the sulfonyl ketone loses a proton and this anion attacks carbon-4 of the nicotinamide ring within NADP, a product of the enzyme. The chemical joining of the sulfonyl ketone with NADP would create bisubstrate analog. The generation of a bisubstrate analog at the active site of the inhibited enzyme, however, is feature of a compound and/or method over conventional methodologies or materials. The sulfonyl group increases the acidity of the proton in question, which facilitates the first step, namely the loss of a proton.

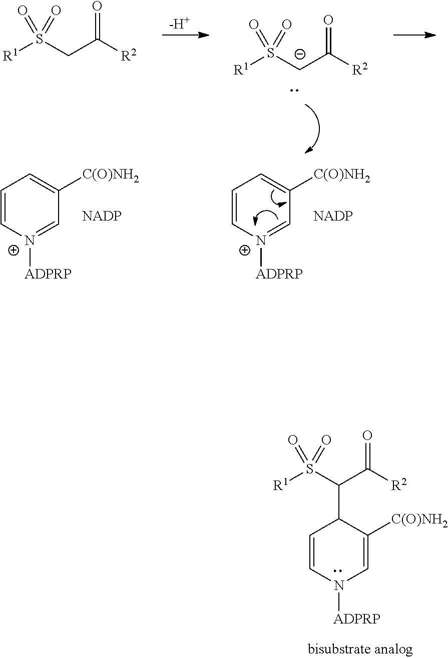

In some example embodiments, enzymes such as ASADH can be inhibited by one or more mechanisms via a sulfonyl acrylamide, for instance. The essential cysteine residue has two possible points of attack, both of which are Michael additions that are similar to those that we propose for vinyl sulfones (e.g. class 1) and acrylamides (e.g. class 3). Therefore, sulfonyl acrylamides are also configured to produce irreversible inhibition. One route of attack may predominate.

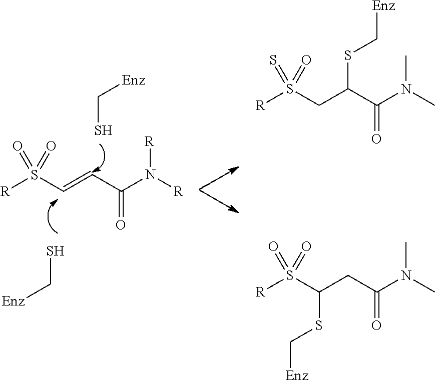

In some embodiments, a method of implementing a compound having selective inactivation energy is provided. In an embodiment, a method comprises providing a compound having a selective inactivation activity, providing an enzyme target, and inactivating the enzyme target with the compound having the selective inactivation activity. In some embodiments, the compound comprises a sulfonyl group. In some embodiments, the compound comprises a vinyl sulfone or a vinyl sulfonamide. In some embodiments, the compound comprises a carboxyl group and/or a nitro group. In some embodiments, the compound exhibits antifungal and/or antibacterial properties. In some embodiments, the enzyme target is a microbial enzyme target, for example ASADH. In some embodiments, when a compound comprises a vinyl sulfone or a vinyl sulfonamide, the vinyl sulfone or vinyl sulfonamide is configured as an isotere of a mixed phosphoric carboxylic anhydride. In some embodiments, the compound can be configured to match a binding pocket of a microbial enzyme target, such as ASADH. In some other embodiments, the compound is a sulfonyl keytone or a sulfonyl acrylamide. In some embodiments, the enzyme target is irreversibly inhibited, or for example irreversibly inhibited by a covalent bonding mechanism.

### EXAMPLES

### Compounds Exhibiting Antimicrobial Activity

Buffers, substrates and most reagents were purchased from Sigma-Aldrich, others known may be used similarly L-aspartate-β-semialdehyde (ASA) was synthesized by ozonolysis of L-allylglycine and subsequently stored in 4 M HCl at −20° C. due to its instability at basic pH. Working solutions of ASA were neutralized immediately prior to addition to the assay mixture.

Diethyl(phenylsulfonyl)methanephosphonate (DPhSUMP) was purchased from Combi-Blocks (San Diego, CA).

The ASADH from Candida albicans (CalASADH) was purified using an ÄKTA chromatography system as previously described (Arachea et al., 2010), with only minor modifications. A nickel-immobilized metal affinity (IMAC) column was used for the purification of the his-tagged enzyme, with an initial buffer wash containing 20 mM imidazole to remove loosely bound proteins before elution with a 20 to 400 mM imidazole gradient. The enzyme activity was assayed at 25° C. by following the production of NADPH at 340 nm in a SpectraMax 190 plate reader (Molecular Devices). Enzyme inhibitors were evaluated by the same assay in the presence of 120 mM CHES buffer, pH 8.6, with 120 mM KCl, 0.3 mM ASA, 1.5 mM NADP, 20 mM phosphate and varying concentrations of each potential inhibitory compound to be tested. The data were fitted to a competitive inhibitor model which corrects for the levels of substrates present relative to their individual Kₘ values.

To carry out the molecular modeling studies the putative vinyl sulfone inactivator structures were created, and the preparation of the ligand and protein files for docking were also created. Each compound was individually docked using Autodock 4.2 into the active site of CalASADH through the formation of a covalent adduct with the active site cysteine nucleophile. Docking studies were performed for a total of 50 runs with a clustering root mean squared distance (RMSD) tolerance value of 2.0 Å and the mean predicted binding energies are reported for the most dominant poses in each cluster (Table 1).

Compounds were prepared according to the following reactions schemes herein, and fall under the formulas described. Reference is made to the following table of compounds:

| Compound | Structure |
|---|---|
| 2-(methylsulfonylvinyl)nitrobenzene | 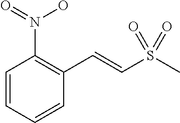 |
| 3-(methylsulfonylvinyl)nitrobenzene | 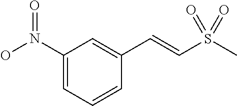 |
| 4-(methylsulfonylvinyl)nitrobenzene | 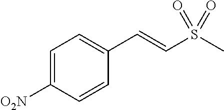 |
| 2-(methylsulfonylvinyl)pyridine | 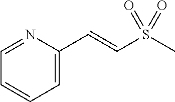 |
| 3-(methylsulfonylvinyl)pyridine | 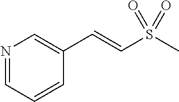 |
| 4-(methylsulfonylvinyl)pyridine | 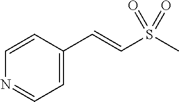 |
| 3-(methylsulfonylvinyl)benzoic acid | 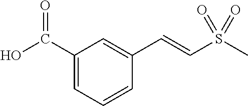 |
| 4-(methylsulfonylvinyl)benzoic acid | 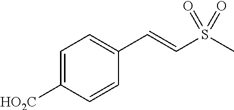 |
| 4-(methylsulfonylvinyl)-1,2-benzenediol | 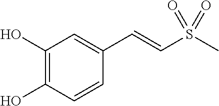 |
| 3-(phenylsulfonylvinyl)acrylic acid | 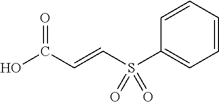 [SMILES: O=C(O)/C=C/S(=O)(=O)c1ccccc1] |
| methylsulfonylvinylbenzene | 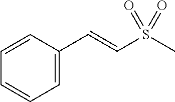 |
| 2-(phenylsulfonylvinyl)pyridine | 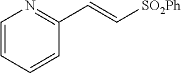 |
| 3-(phenylsulfonylvinyl)pyridine | 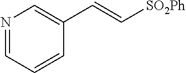 |
| 4-(phenylsulfonylvinyl)pyridine | 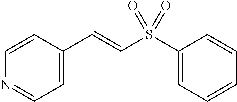 [SMILES: O=S(=O)(/C=C/c1ccncc1)c1ccccc1] |
| 3-(methylsulfonylvinyl)acrylic acid | 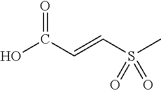 |
| 4-(methylsulfonylvinyl)-benzamide | 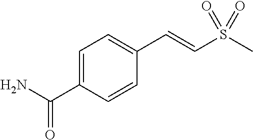 |
| 2-(methylsulfonylvinyl)-1,4-quinol | 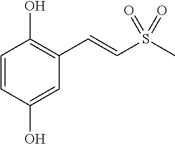 |
| 4-(methylsulfonylvinyl)-acetanilide | 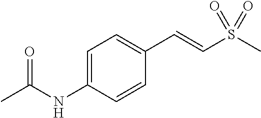 [SMILES: CC(=O)Nc1ccc(/C=C/S(C)(=O)=O)cc1] |
| Trifluoromethylsulfonylvinylbenzene | 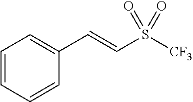 [SMILES: O=S(=O)(/C=C/c1ccccc1)C(F)(F)F] |

The mechanism of nucleophilic attack on vinyl sulfones and the two-step kinetic model for enzyme inactivation. Substitutions at the adjacent R₁ and R₂ positions can be used to alter the reactivity, by changing the electron-withdrawing potential (arrows), and increase the binding selectivity, by changing the specific interactions within the active site pocket.

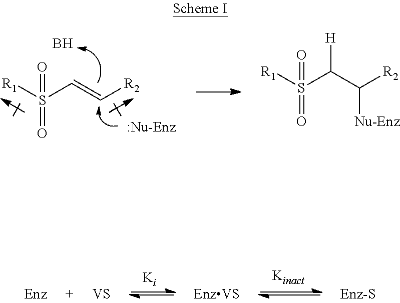

Coupling of aryl aldehydes with diethyl(methylsulfonyl)phosphonates to produce aryl vinyl sulfones where X=CCO₂H, CNO₂, or N, and R₁=methyl or phenyl. For the carboxylates protected as esters deprotection involved base-catalyzed hydrolysis and then neutralization to the acid form.

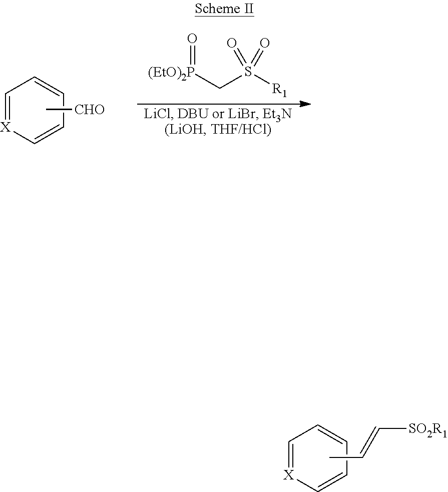

Synthesis of 4-(methylsulfonylvinyl)benzene-1,2-diol (4). 3,4-dihydroxy-benzaldehyde was activated through formation of an imine with either (a) β-alanine or (b) pyrrolidine, followed by coupling with 2-(methylsulfonyl)acetic acid (c) to produce the vinyl sulfone.

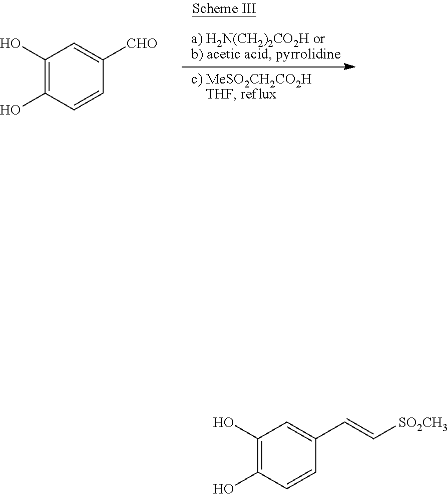

Synthesis of an alkyl vinyl sulfone. Addition of a sulfinate (where R₁=phenyl) with displacement of one bromide ion, followed by elimination of the second bromide to form a β-substituted acrylic acid.

A set of

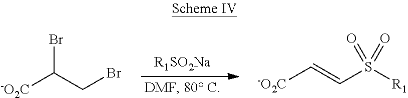

aryl vinyl sulfones were synthesized, primarily through the coupling of β-sulfonyl phosphonates to a series of aryl aldehydes by an adaption of the Horner-Wadsworth-Emmons reaction (Scheme II).

Synthesis of diethyl (methylsulfonyl)methane phosphonate (DMeSUMP). DMeSUMP was synthesized by following a published procedure (Blumenkopf, 1986). Oxone (75.67 mmol, 23.26 g) in 100 mL of water was added to a stirred solution of diethyl (methylthio)methane phosphonate (25.22 mmol, 5.00 g) in 100 mL of ice-cold methanol dropwise over two hours and the reaction was then stirred overnight. The mixture was concentrated by rotary evaporation and the product was extracted with DCM. The combined organic layers were extracted with brine, dried with Na2SO4, and the solvent was removed with rotary evaporation. The yield ranged from 93 to 99%. ¹H NMR (600.2 MHz, CDCl₃) 4.23 (m, 4H), 3.59 (d, J=16.4, 2H), 3.21 (s, 3H), 1.38 (t, J=7.1, 6H). ³¹P NMR (243.0 MHz, CDCl₃) 11.73 (s).

4-(methylsulfonylvinyl)benzoic acid (1). LiBr (6.0 mmol, 0.5211 g), DMeSUMP (6.0 mmol, 1.3811 g), and triethylamine (5.5 mol, 0.77 mL) were dissolved in acetonitrile followed by the dropwise addition of methyl 4-formylbenzoate (5.0 mmol, 0.8208 g). After stirring overnight at rt the reaction was quenched with 0.1 M HCl and extracted with ethyl acetate, washed with water and brine, then dried with MgSO₄. Following solvent removal by rotary evaporation the methyl ester product was eluted from silica using 50:50 hexanes/ethyl acetate. Yield was 79%. ¹H NMR (300 MHz, CDCl₃): 8.22 (s, 1H), 8.12 (d, J=7.6, 1H), 7.68 (d, J=15.8, 1H), 7.66 (d, J=9.1, 1H), 7.53 (t, J=7.8, 1H), 7.01 ppm (d, J=15.4 ppm) 3.96 (s, 3H), 3.05 (s, 3H). Hydrolysis of the methyl ester was carried out by dissolving in 60:40 THF/water, adding LiOH and stirring for 75 min. The solvent was removed, the product was dissolved in water, acidified with HCl, and the precipitate was collected by filtration. Additional product was obtained by extracting the filtrate with ethyl acetate. The overall yield was 90%. ¹H NMR (300 MHz, d₆-DMSO): 13.20 (s, 1H), 7.99 (d, J=8.2, 2H), 7.84 (d, J=8.2, 2H), 7.65 (d, J=15.6, 1H), 7.57 (d, J=15.6, 1H), 3.13 (s, 3H).

3-(methylsulfonylvinyl)benzoic acid (13). LiCl (11.32 mmol, 0.48 g) and DMeSUMP (7.0 mmol, 1.61 g) were dissolved in acetonitrile and neat DBU (5.8 mmol, 0.87 mL) was added. Methyl 3-formylbenzoate (5.8 mmol, 0.95 g) in acetonitrile was added dropwise and the reaction stirred for two days. The solvent was removed with rotary evaporation, and the reaction was quenched with saturated ammonium chloride. The crude product was extracted with DCM and the organic phase was dried with MgSO₄. The purified methyl ester was eluted from silica using ethyl acetate. Yield was 26%. ¹H NMR (300 MHz, CDCl₃): 8.22 (s, 1H), 8.12 (d, J=7.6, 1H), 7.68 (d, J=15.8, 1H), 7.66 (d, J=9.1, 1H), 7.53 (t, J=7.8, 1H), 7.01 ppm (d, J=15.4 ppm) 3.96 (s, 3H), 3.05 (s, 3H).

Methyl 3-[2-(methylsulfonyl)vinyl]benzoate (1.13 mmol, 0.2589 g) was dissolved in 3:2 THF/water, LiOH (2.27 mmol, 0.09518 g) was added, and the reaction was stirred for 1 h. The organic solvent was removed by rotary evaporation, the hydrolyzed product was acidified with HCl and the precipitate was collected by filtration and dried under vacuum. The filtrate was extracted ethyl acetate and the organic phase dried with MgSO₄ to provide the purified product. The yield was 61%. ¹H NMR (600.2 MHz, d₆-DMSO): 13.04 (s, 1H), 8.23 (s, 1H), 7.99 (d, J=7.7, 1H), 7.94 (d, J=7.7, 1H), 7.58 (t, J=7.8, 1H), 7.58 (d, J=15.7, 1H), 7.56 (d, J=15.7, 1H), 3.09 (s, 3H). ¹³C NMR (150.9 MHz, d₆-DMSO): 167.17, 140.65, 133.50, 133.21, 132.26, 131.81, 130.26, 129.84, 129.61, 43.07.

Diethyl trifluoromethylsulfonylmethylphosphonate. A 1.6 M solution of n-BuLi (7.23 mmol, 4.5 mL) in hexanes was added dropwise to diethyl methylphosphonate (1.02 g, 6.68 mmol) in diethyl ether and the mixture was stirred at −72° C. for 1 h. Trifluoromethanesulfonic anhydride (3.29 mmol, 0.55 mL) was added dropwise and the reaction was stirred for 1 h, followed by quenching with 5% HCl. The aqueous layer was thrice extracted with diethyl ether and the combined organic layers were extracted against brine and dried with Na₂SO₄. The purified product was eluted from silica gel using 40:60 acetone/hexanes with a yield of 20%. ¹H NMR (300.1 MHz, CDCl₃) 4.32 (m, 4H), 3.81 (d, J=18.1, 2H) 1.43 (t, J=7.1, 6H). ¹³C NMR (75.5 MHz, CDCl₃) 119.1 (qd, J=327.0; J=8.6), 46.4 (d, J=133.6), 16.2 (d, J=6.3). ³¹P NMR (121.5 MHz, CDCl₃) 7.6 ppm (s). ¹⁹F NMR (282.4 MHz, CDCl₃)-78.9 ppm (s).

Trifluoromethylsulfonylvinylbenzene (7). Diethyl trifluoromethylsulfonylmethylphosphonate (0.180 g, 0.63 mmol) was dissolved in a 2 mL aliquot of dry THF. Potassium tert-butoxide (0.075 g, 0.67 mmol) was added and then benzaldehyde (0.075 g, 0.70 mmol) in 2 mL of THF was added dropwise and the reaction was refluxed for 15 h with monitoring by TLC. After completion the reaction was quenched with saturated ammonium chloride and extracted with DCM. The organic layers were dried with MgSO₄ and the volatiles were removed with rotary evaporation. The purified product was eluted from silica using 50:50 toluene/hexanes with an overall yield of 55%. ¹H NMR (300.1 MHz, CDCl₃) 7.90 (d, J=15.5, 1H), 7.66-7.46 (m, 5H), 6.84 (d, J=15.5) 1H). ¹³C NMR (75.5 MHz, CDCl₃) 153.80, 133.22 131.19, 129.55, 129.49, 119,72 (q, J=325), 116.67. ¹⁹F NMR (282.4 MHz, CDCl₃)-78.73 (s).

Coupling Reaction. The following compounds were synthesized from the same coupling reaction (Scheme II). LiCl was dissolved in acetonitrile followed by the addition of either DMeSUMP or DPhSUMP. One equivalent of 1,8-diazabicyclo[5.4.0]undec-7-ene (DBU) was then added to facilitate extraction of an α-proton adjacent to phosphorus, followed by the dropwise addition of each aldehyde. The reaction mixtures were stirred at rt with progress monitored by TLC (50:50 ethyl acetate/hexane) until the aldehyde was completely consumed (typically within two hours). Each reaction was then quenched with saturated ammonium chloride solution and rotary evaporated to remove the solvent. The products were extracted with four washings of DCM (DCM), dried with magnesium sulfate, and the DCM was removed by rotary evaporation to produce the crude product. Elution from silica gave the final purified products.

4-(methylsulfonylvinyl)nitrobenzene (2). DMeSUMP (5.26 mmol, 1.212 g) was added to the LiCl (0.3506 g, 8.27 mmol) solution, followed by the addition of DBU (4.40 mmol, 0.68 mL) and the dropwise addition of 4-nitrobenzaldehyde (4.54 mmol, 0.6854 g) in acetonitrile. The reaction mixture was quenched with saturated ammonium chloride, and the volatiles were removed by rotary evaporation. The crude product was extracted into DCM, and the organic layer was dried and removed with rotary evaporation. The purified product was eluted from silica using 67:33 ethyl acetate/hexanes. The yield was 48%. ¹H NMR (300 MHz, CDCl₃): 8.30 ppm (m, 2H), 7.69 ppm (d, 2H), 7.69 ppm (d, J=15.4, 1H), 7.06 ppm (d, J=15.5, 1H), 3.08 ppm (s, 3H). ¹³C NMR (151 MHz, CDCl₃): 149.31, 140.94, 138.14, 130.75, 129.19, 124.34, 43.05.

2-(methylsulfonylvinyl)pyridine (3). LiCl (6.6 mmol, 0.28 g) was dissolved in a 35-mL portion of acetonitrile. DMeSUMP (4.3 mmol, 1.00 g) and then neat DBU (3.6 mmol, 0.54 mL) were added, followed by the dropwise addition of 2-pyridine carboxaldehyde (3.6 mmol, 0.39 g) in acetonitrile. The reaction was quenched and the volatiles were removed by rotary evaporation. The crude product was extracted with DCM, the organic layer was dried, and the solvent removed by rotary evaporation. The purified product was eluted from silica with ethyl acetate. Yield was 41%. ¹H NMR (600 MHz, CDCl₃): 8.66 (d, J=4, 1H); 7.75 (m, 1H), 7.61 (d, J=14.9, 1H); 7.52 (d, J=14.9, 1H); 7.41 (d, J=7.7, 1H); 7.32 (dd, J=7, J=5, 1H); 3.04 (s, 3H). ¹³C NMR (151 MHz, CDCl₃): 150.88, 150.40, 142.02, 136.99, 130.99, 125.42, 125.08, 43.05.

4-(phenylsulfonylvinyl)pyridine (5). LiCl (0.215 g, 5.1 mmol) was dissolved in acetonitrile, and DPhSUMP (0.9993 g, 3.4 mmol) in acetonitrile was added. DBU (0.450 mL, 3.0 mmol) in acetonitrile was added, and 4-pyridinecarboxaldehyde (0.295 g, 2.8 mmol) was added dropwise with stirring. The reaction was quenched and the volatiles were removed with rotary evaporation. The crude product was extracted into DCM, which was dried with MgSO₄ and removed with rotary evaporation. The purified product was eluted from silica using 75:25 to 80:20 ethyl acetate/hexanes. The yield was 96%. ¹H (300 MHz, CDCl₃): 8.70 (dd, J=4.5, 1.5, 2H), 7.99 (d, J=6.9, 2H), 7.72-7.58 (m, 3H), 7.64 (d, J=15.9, 1H), 7.35 (dd, J=4.8, 1.8, 2H), 7.06 (d, J=15.6, 1H). ¹³C (75.5 MHz, CDCl₃): 150.84, 139.75, 139.60, 139.38, 133.90, 132.22, 129.56, 127.95, 122.06.

Methylsulfonylvinylbenzene (6). LiCl (7.07 mmol, 0.30 g) was dissolved in acetonitrile and DMESUMP (4.26 mmol, 0.98 g) in acetonitrile and DBU (3.68 mmol, 0.55 mL) were added. Benzaldehyde (3.62 mmol, 0.3841 g) in acetonitrile was added dropwise with stirring. The reaction was quenched and volatiles were removed by rotary evaporation. The crude product was extracted into DCM, and the solvent dried with MgSO₄ and removed with rotary evaporation. The purified product was eluted from silica using 50:50 ethyl acetate/hexanes. The yield was 56%. ¹H (300 MHz, CDCl₃): 7.66 (d, J=15.6, 1H), 7.56-7.45 (m, 5H), 6.94 (d, J=15.3, 1H), 3.06 (s, 1H). ¹³C (75.5 MHz, CDCl₃): 144.10, 132.10, 131.44, 129.20, 128.59, 126.17, 43.33.

2-(Phenylsulfonylvinyl)pyridine (8). LiCl (0.280 g, 6.6 mmol) was dissolved in acetonitrile, and DPhSUMP (1.0330 g, 3.5 mmol) in acetonitrile and DBU (0.54 mL, 3.6 mmol) were added. 2-pyridinecarboxaldehyde (0.313 g, 2.9 mmol) in acetonitrile was added dropwise with stirring. The reaction was quenched and the volatiles were removed by rotary evaporation. The crude product was extracted into DCM, and the solvent dried with MgSO₄ and removed with rotary evaporation. The purified product was eluted from silica using 50:50 ethyl acetate hexanes. The yield was 96%. ¹H (300 MHz, CDCl₃): 8.64 (d, J=3.9, 1H), 7.99 (dd, J=7.2, 1.5, 1H), 7.76 (td, J=7.8, 1.8, 1H), 7.68 (d, J=15.0, 1H), 7.48 (d, J=15.0, 1H), 7.44 (d, J=8.4, 1H), 7.32 (ddd, J=7.5, 4.8, 0.9, 1H). ¹³C (75.5 MHz, CDCl₃): 151.05, 150.34, 140.53, 140.26, 137.05, 133.56, 131.56, 129.37, 127.91, 125.50, 125.02.

4-(methylsulfonylvinyl)pyridine (9). LiCl (0.214 g, 5.1 mmol) was dissolved in acetonitrile, and DMeSUMP (0.78 g, 3.4 mmol) in acetonitrile was added. DBU (0.425 mL, 2.8 mmol) in acetonitrile was added, and 4-pyridinecarboxaldehye (0.3110 g, 2.9 mmol) in acetonitrile was added dropwise with stirring. The reaction was quenched and the volatiles were removed with rotary evaporation. The crude product was extracted into DCM, which was dried with MgSO₄ and removed with rotary evaporation. The purified product was eluted from silica using ethyl acetate. The yield was 79%. ¹H (300 MHz, CDCl₃): 8.74 (dd, J=4.5, 1.5, 2H), 7.60 (d, J=15.3, 1H), 7.39 (dd, J=4.5, 1.5, 2H), 7.13 (d, J=15.6, 1H), 3.08 (s, 3H). ¹³C (75.5 MHz, CDCl₃): 150.36, 141.25, 139.31, 130.95, 122.07, 43.02.

3-(methylsulfonylvinyl)pyridine (10). LiCl (5.7 mmol, 0.24 g) was dissolved in acetonitrile at room temperature. DMeSUMP (5.0 mmol, 1.16 g) in acetonitrile and neat DBU (0.50 mL, 3.3 mmol) were added. Pyridine 3-carboxaldehyde (4.7 mmol, 0.44 mL) in acetonitrile was added dropwise. The reaction was run overnight then quenched with saturated ammonium chloride. The crude product was dissolved in a small volume of water and extracted with DCM. The combined organic layers were dried with MgSO₄, and the volatiles were removed with rotary evaporation. The purified product was eluted from silica using 96:4 ethyl acetate/ethanol. Yield was 82%. ¹H NMR (600 MHz, CDCl₃), 8.77 (s, 1H), 8.67 (d, J=4.0, 1H), 7.82 (d, J=7.8, 1H), 7.64 (d, J=15.5, 1H), 7.37 (dd, J=7.7 and 4.7, 1H), 7.00 (1d, J=15.5 Hz, 1H), 3.04 (s, 3H). ¹³C NMR (151 MHz, CDCl₃): 152.01, 149.91, 140.39, 134.76, 128.73, 128.12, 123.82, 43.13.

3-(Phenylsulfonylvinyl)pyridine (11). LiCl (0.221 g, 5.2 mmol) was dissolved in acetonitrile, and DPhSUMP (1.01 g, 3.5 mmol) in acetonitrile was added. DBU (0.44 mL, 2.9 mmol) was added; then 3-pyridinecarboxaldehyde (0.3231 g, 3.0 mmol) was added dropwise with stirring. The reaction was quenched and the volatiles were removed by rotary evaporation. The crude product was extracted into DCM, and the organic layer was dried with MgSO₄ and the volatiles were removed with rotary evaporation. The purified product was eluted from silica with 75:25 ethyl acetate/hexanes. The yield was 85%. ¹H (300 MHz, CDCl₃): 8.75 (d, J=2.1, 1H), 8.66 (dd, J=4.8, 1.5, 1H), 8.00-7.97 (m, 2H), 7.81 (dt, J=7.8, 2.1, 1H), 7.71 (d, J=15.6, 1H), 7.71-7.55 (m, 3H), 7.36 (dd, J=7.8, 4.8, 1H), 6.98 (d, J=15.5, 1H). ¹³C (75.5 MHz, CDCl₃): 151.59, 150.04, 140.12, 138.77, 134.79, 133.74, 129.60, 129.50, 128.30, 127.84, 123.86.

3-(methylsulfonylvinyl)nitrobenzene (12). LiCl (6.62 mmol, 0.2808 g) was dissolved in acetonitrile. A solution of DMeSUMP (4.34 mmol, 1.0000 g) was added, followed by DBU (3.62 mmol, 0.54 mL). A solution of 3-formylnitrobenzene (3.62 mmol, 0.5470 g) was added dropwise, the reaction was quenched and the volatiles were removed by rotary evaporation. The crude product was extracted with DCM. The organic layer was dried with MgSO₄, and the solvent removed by rotary evaporation. The purified product was eluted from silica using 50:50 hexanes/ethyl acetate. Yield was 72%. ¹H NMR (300 MHz, CDCl₃): 8.40 (s, 1H), 8.32 (d, J=8.4 Hz, 1H), 7.83 (d, J=7.2, 1H), (d, J=15.3, 1H), 7.64 (m, 1H), 7.08 (d, J=15.6, 1H), 3.08 (s, 3H). ¹³C NMR (151 MHz, CDCl₃): 148.91, 141.01, 134.08, 134.01, 130.29, 129.88, 125.48, 122.76, 43.10.

2-(methylsulfonylvinyl)nitrobenzene (14). LiCl (6.7 mmol, 0.2840 g) was dissolved in acetonitrile, and DMeSUMP (4.38 mmol, 1.0086 g) was added. After DBU (3.6 mmol, 0.54 mL) was added, 2-nitrobenzaldehyde (3.6 mmol, 0.55 g) was added dropwise and the reaction mixture was stirred overnight. After quenching the volatiles were removed with rotary evaporation. The crude product was extracted into DCM, and the solvent was dried with MgSO₄ and removed with rotary evaporation. The purified product was eluted from silica using 50:50 to 75:25 ethyl acetate/hexanes. The yield was 98%. ¹H NMR (300 MHz, CDCl₃): 8.14 ppm (d, J=15.4, 1H), 8.13 ppm (m, 1H), 7.74-7.57 ppm (m, 3H), 6.84 ppm (d, J=15.4, 1H), 3.09 ppm (s, 3H). ¹³C NMR (151 MHz, CDCl₃): 148.11, 140.53, 133.85, 131.54, 131.15, 129.55, 128.84, 125.20, 43.02.

4-(methylsulfonylvinyl)-1,2-benzenediol (4). The catechol-containing vinyl sulfone was synthesized by conversion of 3,4-dihydroxybenzaldehyde to its imine, followed by coupling with 2-(methylsulfonyl)acetic acid (Scheme III). This synthesis was run under two different conditions. For the first synthesis methylsulfonylacetic acid (7.2 mmol, 1.00 g) was mixed with 0.5012 g (3.63 mmol) of 3,4-dihydroxybenzaldehyde in a 2:1 ratio, followed by the addition of 3-aminopropionic acid (7.2 mmol, 0.64 g) in tetrahydrofuran with refluxing. In the second synthesis methylsulfonylacetic acid (7.2 mmol, 1.00 g) and 3,4-dihydroxybenzaldehyde (7.2 mmol, 1.00 g) were mixed in a 1:1 ratio, acetic acid (4.0 mmol, 0.23 mL) and pyrrolidine (4.0 mmol, 0.33 mL) were then added and heated to reflux in THF. The stirred reactions proceeded for 1 to 2 days, the combined organic layers were extracted against brine and dried with MgSO₄, and the solvent removed by rotary evaporation. The two crude products were extracted against saturated aqueous sodium bisulfite to remove unreacted aldehyde and then combined. The aqueous phase was extracted using 60:40 ethyl acetate/hexanes and the purified product was eluted from silica using ethyl acetate. The overall yield was 20%. ¹H NMR (600 MHz, CDCl₃): 7.50 (d, J=15.4, 1H), 7.04 (s, 1H), 7.02 (d, J=8.2, 1H), 6.90 (d, J=8.2, 1H), 6.72 (d, J=15.4, 1H), 5.43 (1s, 1H), 5.20 (s, 1H), 3.00 (s, 3H). ¹³C NMR (151 MHz, d₆-DMSO): 149.25, 146.16, 142.38, 124.76, 124.32, 122.22, 116.30, 115.74, 43.48.

An alkyl vinyl sulfone inactivator was synthesized by the addition of a substituted sulfonic acid to a dibromo acid ester and subsequent deprotection of the product (Scheme IV).

3-(phenylsulfonyl)acrylic acid (15). Sodium phenylsulfinate (3.75 mmol, 0.6155 g) and 2,3-dibromopropanoic acid (2.49 mmol, 0.5769 g) were dissolved in dimethylformamide. The reaction was heated to 80° C. for 24 h with stirring. The reaction mixture was dissolved in 15 mL of water and extracted with ethyl acetate. The combined organic layers were washed with brine and dried with MgSO₄. Volatiles were removed with rotary evaporation, and DMF was removed using a SpeedVac. The purified product was eluted from silica using 0-5% ethanol in DCM. The yield was 9.7%. ¹H NMR (400 MHz, CDCl₃): 7.94 (m, 2H), 7.71 (m, ¹H), 7.61 (m, 2H), 7.40 (d, J=15.2 Hz, 1H), 6.83 (d, J=15.2 Hz, 1H) ppm. ¹³C NMR (101 MHz, (CDCl₃): 167.06, 145.02, 138.10, 134.61, 129.86, 129.73, 128.43.

To examine the possibility of enzyme inactivation each vinyl sulfone compound was incubated in a defined concentration range with 30 μg/ml of CalASADH, with the inhibitor concentrations grouped around their determined Kᵢ values. Aliquots (10 μl) were removed from the enzyme-inhibitor reaction mixture at various times ranging from 30 secs to 60 min, with most compounds examined from 1 to 10 min at 2 min intervals. Each aliquot was then added to a 190 μl of the same assay mixture as described above for the inhibition studies to measure the residual enzyme activity. The natural log of the ratio of the rate in the presence of inhibitor over the control rate in the absence of inactivator (In v/vₒ) was plotted against the incubation time to determine the observed rate (k_{obs}) at each inactivator concentration. A plot of these observed rates vs. the inactivator concentrations will yield the rate of inactivation (k_{inact}) for each compound that irreversibly inactivates the enzyme. This value can be obtained either from a linear least squares fit to a reciprocal plot of these values or from a hyperbolic fit to the direct plot of these values, with the non-linear hyperbolic fit being the preferred method.

The following example compounds further exemplify embodiments of the technology described herein, which can act as inhibitors and/or inactivators of microbial compositions, for instance ASADH or homoserine dehydrogenase. As will be appreciated, these compounds, and those described previously, incorporate a core functional group such as vinyl sulfone and/or an acrylamide.

Some example compounds fall under a classification of an amino acid analog, and are provided as follows:

| Compound | Structure |
|---|---|
| 3-(S-methylsulfonylvinyl)- alanine hydrochloride | 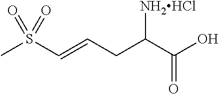 |
| 3-(benzylsulfonylvinyl)- alanine hydrochloride | 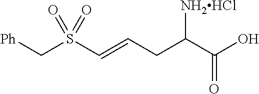 |
| 3-acrylamidoalanine hydrochloride | 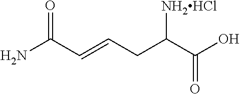 |

These amino acid analogs may be synthesized in accordance with various techniques and schemes provided herein. Two are vinyl sulfones and one is an acrylamide. Accordingly, the core functional group of compounds described herein can be at least one of a vinyl sulfone and/or an acrylamide. The vinyl sulfones were tested against C. auris ASADH as irreversible inhibitors. S-benzylsulfonylvinylalanine has an inhibitory constant of 300 nM, and S-methylsulfonylvinylacrylamide had an inhibitory constant of 100 nM.

Some example compounds in accordance with various aspects of the present technology can be determined to fall under a classification of aromatic compounds, and are provided as follows:

| Compound | Structure |
|---|---|
| 2-(S-methylsulfonylvinyl)phenyl- ortho-glucopyranoside | 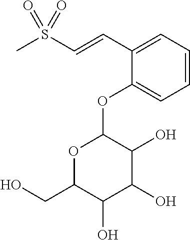 [SMILES: CS(=O)(=O)/C=C/c1ccccc1OC1OC(CO)C(O)C(O)C1O] |
| 2-(S-methylsulfonylvinyl)quinoline | 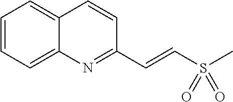 [SMILES: CS(=O)(=O)/C=C/c1ccc2ccccc2n1] |
| 4-(S-methylsulfonylvinyl)acetamide | 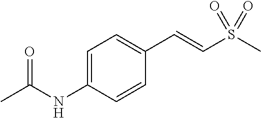 [SMILES: CC(=O)Nc1ccc(/C=C/S(C)(=O)=O)cc1] |
| 4-(S-methylsulfonylvinyl)benzamide | 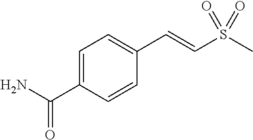 |
| 2-(S-methylsulfonylvinyl)1,4- dihydroxybenzene | 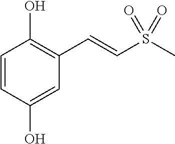 |
| 4-(S-(2-pyridyl)sulfonylvinyl)pyridine | 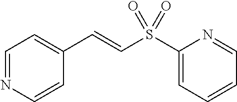 [SMILES: O=S(=O)(/C=C/c1ccncc1)c1ccccn1] |
| 4-acrylamidopyridine | 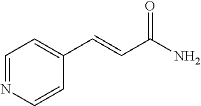 [SMILES: NC(=O)/C=C/c1ccncc1] |
| 5-(S-methylsulfonylvinyl)isoquinoline | 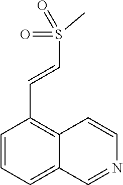 [SMILES: CS(=O)(=O)/C=C/c1cccc2cnccc12] |

These aromatic compounds (i.e. aromatic vinyl sulfones) may be synthesized in accordance with various techniques and schemes provided herein. As will be appreciated, at least one compound is also a glucopyranoside, which was designed for better transport across microbial membranes. Further, an aromatic acrylamide was synthesized. Some of these compounds have been tested against ASADH. One compound, 4-(S-(2-pyridyl)sulfonylvinyl)pyridine, was synthesized to test the effect of putting an electron-withdrawing group on sulfur. It resembles two previously synthesized compounds, one of which showed modest but broad activity in disk diffusion assays, described below.

Some example compounds in accordance with aspects of the present technology fall under a classification of vinyl sulfone carboxymides, and are provided as follows:

| Compound | Structure |
|---|---|
| 3-S-Methylsulfonylacrylamide | 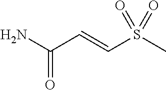 |
| 3-S-Phenylsulfonylacrylamide | 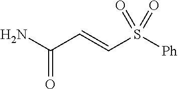 [SMILES: NC(=O)/C=C/S(=O)(=O)c1ccccc1] |

In the above examples, these compounds of the form RSO₂CH═CHC(O)NH₂ were synthesized, where R=methyl or phenyl. These two compounds, for example, differ from the other vinyl sulfones in that they have a strongly electron-withdrawing group on the other carbon of the double bond. This distinction is a matter of degree with aromatic rings that are somewhat electron withdrawing.

The following disk diffusion assays were performed with example compounds described above to assess the ability of compounds to inhibit growth of microorganism. A compound that inhibits its target enzyme may perform poorly for a number of reasons, including low solubility in water or slow rate of transport across a microbial membrane.

In the following experimental examples, three aromatic compounds were tested and are shown to have modest to moderate effects, for example against enzymes tested, in disk diffusion assays:

| Compound | S. aureus | K pneumoniae | C. albicans |
|---|---|---|---|
| 4-MSV-pyridine | medium | medium | medium |
| 2-MSV-dihydroxbenzene | medium | weak | |
| 2-MSV-quinoline | | | weak |

In the following experiments vinyl sulfone carboxamide compounds were tested: 3-S-Methylsulfonylacrylamide and 3-S-Phenylsulfonylacrylamide. These compounds were good inhibitors against a gram-negative and a gram-positive bacterium:

| | S. aureus | K pneumoniae | C. albicans |
|---|---|---|---|
| MeSAm | strong | strong | |
| PhSAm | strong | strong | |

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention. Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A method comprising:
providing a compound having a selective inactivation activity;
providing an enzyme target; and
inactivating the enzyme target.

2. The method of claim 1, wherein the compound comprises a sulfonyl group.

3. The method of claim 1, wherein the compound comprises a vinyl sulfone or a vinyl sulfonamide.

4. The method of claim 1, wherein the compound comprises at least one of a carboxyl group and a nitro group.

5. The method of claim 1, wherein the compound exhibits at least one of antifungal and antibacterial properties.

6. The method of claim 1, wherein the enzyme target is a microbial enzyme target.

7. The method of claim 1, wherein the microbial enzyme target is ASADH.

8. The method of claim 3, wherein the vinyl sulfone or the vinyl sulfonamide is configured as an isostere of a mixed phosphoric carboxylic anhydride.

9. The method of claim 7, wherein the compound is configured to match a binding pocket of ASADH.

10. The method of claim 1, wherein the compound is a sulfonyl keytone or a sulfonyl acrylamide.

11. The method of claim 1, wherein the enzyme target is irreversibly inhibited.

12. The method of claim 11, wherein the enzyme target is irreversibly inhibited by covalent bonding.
